(19) United States
(12) Patent Application Publication
(10) Pub. No.: US 2026/0250226 A1
(43) Pub. Date: Aug. 27, 2026

(54) METHOD OF ISOLATION AND PURIFICATION OF XANTHOHUMOL

(71) Applicant: INNOX Sp. z o.o., Bialystok (PL)

(72) Inventors: Henryk GRUZA, Warszawa (PL); Kajetan DABROWA, Warszawa (PL); Mariusz GUJSKI, Warszawa (PL); Aran DEVRIM, Far Hills, NJ (US); Lukasz ZYBACZYNSKI, Konstancin-Jeziorna (PL)

(73) Assignee: INNOX Sp. z o.o., Bialystok (PL)

(21) Appl. No.: 18/992,322

(22) PCT Filed: Jul. 7, 2023
(86) PCT No.: PCT/PL2023/000034

§ 371 Date: Jan. 8, 2025

(30) Foreign Application Priority Data
Jul. 8, 2022 (PL) P.441690

(51) Int. Cl. C07C 45/79; C07C 45/80

(52) U.S. Cl. CPC C07C 45/79; C07C 45/80

(57) ABSTRACT

The present invention is a method of isolating and purifying a plant-derived biologically active substance (xanthohumol) from the plant material of hops and from the by-product formed in the process of extracting hops with supercritical carbon dioxide.

The present invention is related to a method of isolating and purifying a plant-derived biologically active substance (xanthohumol) from the plant material of hops and from the by-product formed in the process of extracting hops with supercritical carbon dioxide.

Xanthohumol (Xanthohumol, 2′,4,4′-Trihydroxy-6′-methoxy-3′-(3-methylbut-2-en-1-yl) chalcone, CAS: 6754-58-1, abbreviated Xn) is a substance of natural origin in the prenylated chalcone family. Xn is found in the female inflorescences of Humulus lupulus (hop cones). The compound is also found in beer, giving it a bitter taste.

Description of WO 2014/016409 discloses a pharmaceutical composition containing a therapeutic amount of xanthohumol and a roasted extract, advantageously a hop extract containing xanthohumol for treating and preventing cancer. The roasted extract is selected from a cold or hot extract of coarse ground or unground roasted malt, cereal, coffee, and cocoa.

From another description EP 1761245 B, it is known how to produce a roasted extract containing xanthohumol from the products of roasting cereals, cereal malt, or coffee and hop extract containing Xn, and its use in the production of beverages and foodstuffs.

The description of CN 101440029 discloses a method of extracting xanthohumol from powdered hops and supercritical carbon dioxide (scCO₂). The collected hop raffinate is subjected to ultrasonic extraction with alcohol, and the extraction solution is condensed, mixed with diatomaceous earth, and eluted with mixtures of alcohol and water, obtaining the final product xanthohumol after filtration and condensation treatment at low temperature.

However, the processes described in the literature for the extraction and isolation of xanthohumol from plant material have many drawbacks and disadvantages, such as:
i) the need to use large quantities of organic solvents, especially solvents with high toxicity (dichloromethane, n-hexane, chloroform-e.g., CN 101440029, CN 103524321);
ii) the need to use large quantities of expensive materials, such as silica gel (silica gel);
iii) (iii) irreversible decomposition of xanthohumol to by-products (mainly isomeric isoxanthohumol, CAS: 521-48-2) and to polymeric oxidative degradation products (extraction using aqueous solutions of strong bases e.g., NaOH, KOH);
iv) low process efficiency and scalability (e.g., Countercurrent chromatography technique);
v) inability to obtain large quantities of high-purity xanthohumol.

In addition, the methods described for obtaining xanthohumol by chemical synthesis are inefficient and not very scalable. For example, the paper J. Nat. Prod. 2007, 70, 1507-1509 describes how to synthesize small amounts of Xn (<0.1 g) by six-step chemical synthesis with a total yield of 10%. Moreover, the final purification step does not allow economically viable preparation of large quantities of xanthohumol with high pharmaceutical purity.

Considering the issues presented, it is evident that there is a need for new methods of isolating xanthohumol from hop plant material and by-products, which will be devoid of the above disadvantages and inconveniences.

The aim of the present invention is to obtain xanthohumol of high pharmaceutical purity from plant material. According to the invention, the developed method offers a cost-effective technology for extracting and purifying xanthohumol from widely available plant material on a semi-technical and industrial scale with a low environmental impact.

The present invention is a new method of isolating and purifying xanthohumol by extraction from plant material, which consists of the following:
an appropriate amount of plant material is weighed, and then a measured amount of mixture A is added, where the contents of the extractor are subjected to a continuous extraction process under an inert gas atmosphere at 20-90° C. for 2-96 h, mixture A and the soluble fractions of the plant material are taken through a suitable ceramic flow filter system for evaporation, and the evaporated solvent is returned to the extractor, and the solid residue X1 is waste;
mixture A is removed from the extractor, and mixture B is added to the solid mass, where the extractor contents are subjected to a continuous extraction process under an inert gas atmosphere at 20-90° C. for 0.5-24 h; mixture B and the soluble fractions of the plant raw material are taken through a suitable ceramic flow filter system for evaporation, mixture B is evaporated and the solid residue X2 is the raw product for further processing after the extraction is completed, the extractor contents are swept to a filter press and the filtrate after the evaporation of mixture B is attached to X2, the solid residue from the filter press is the waste X3;
the weighed amount of X2 is dissolved in mixture C to obtain a concentration of Xn 0.005-0.5 mol/l, the resulting solution of X2 in mixture C is subjected to a process of successive single or multiple extractions against mixture D with a concentration of 0.005-1.0 mol/l, mixture E with a concentration of 0.005-0.2 mol/l, mixture F with a concentration of 0.005-1.0 mol/l and mixture G with a concentration of 0.05-1.0 mol/l to obtain solution H, the extraction process is carried out at a temperature of 10-50° C., preferably 10-30° C., in an inert gas atmosphere;
in this step of isolation and purification of Xn, a reversible process of formation of very water-soluble Xn salts (phenolates) is used, which, after neutralization with an aqueous acid solution from the D-mixture, results in a suspension of Xn in water, the H-solution is set to an Xn content of 0.01-0.1 mol/l (preferentially 0.05-0.10 mol/l), after which the H solution is subjected to an alkaline-acid extraction process in the flow using a liquid-liquid separator, two streams of solutions are fed into the flow reactor-the H solution and the I mixture, the flow reactor is connected directly to the liquid-liquid separator, the organic stream coming out of the separator is waste X4, and the water stream is subjected to neutralization with mixture D, the extraction process is carried out at a temperature of 2-35° C., preferentially 20-25° C., in an inert gas atmosphere;
a suspension of Xn in water is obtained, which is subjected to (a) filtration, (b) centrifugation, or (c) extraction with mixture C; the organic phase after the extraction process is washed with mixture G, preferably sodium chloride or sodium sulfate, of 0.05-1.0 mol/l, preferably 0.1-0.2 mol/l, and then dried with molecular sieves or anhydrous sodium sulfate, magnesium sulfate or their mixture of the said drying agents; after the drying agent is drained off, solution J is obtained (Xn content in dry weight is 80-90% by weight); after filtration (a) or centrifugation (b), the Xn precipitate is dissolved in mixture C and subjected to analogous processes as after process (c);
a measured amount of Mixture A is added to solution J in which Xn is sparingly soluble (preferentially C7-C8 saturated hydrocarbons, preferably n-heptane). The volatile fractions are evaporated until Xn precipitation is initiated, the precipitated Xn is drained and washed with Mixture A at 0-40° C. (preferably 15-25° C.), the filtrate constitutes waste X5, the precipitate is macerated with Mixture C, the insoluble precipitate is re-drained, and the filtrate is waste X6, the precipitate is dried at 15-25° C. under reduced pressure, the dry precipitate is dissolved in mixture B and the volatile fractions are evaporated to dryness under reduced pressure at 15-35° C., preferably 25-30° C., after vacuum drying at 15-25° C., the final product is obtained in the form of a light yellow powder (Xn content ≥99.5%, as determined by HPLC).

A method where the extraction in an inert gas atmosphere is advantageously at 25-50° C. for 6-36 h.

A method where mixture A is saturated, unsaturated, or cyclic C₅-C₈ hydrocarbons, toluene, xylenes (ortho-xylene, meta-xylene, para-xylene, and mixtures thereof).

A method where the residue X1, X2, or X3 is xanthohumol.

A method where mixture B is a polar organic solvent or a mixture of polar organic solvents, i.e.:
aliphatic alcohols with the general formula ROH, where R=C₁-C₄ aliphatic group, in particular methyl alcohol (MeOH), ethyl alcohol (EtOH), 2-propanol (i-PrOH),
esters with the general formula R₁CO₂R₂, where R₁/R=saturated and unsaturated aliphatic C₁-C₄ hydrocarbons and R₂=aromatic hydrocarbons or aromatic-aliphatic hydrocarbons (e.g. PhCH₂—), in particular ethyl acetate,
liquid ethers with the general formula R₁OR₂ where R₁/R₂=saturated and unsaturated, acyclic and cyclic aliphatic hydrocarbons C₁-C₄, in particular diethyl ether (Et₂O), diisopropyl ether (i-Pr₂O), tert-butyl methyl ether (t-BuOMe), and 2-methyl tetrahydrofuran (2-MeTHF),
liquid polar aprotic solvents such as N,N-dimethylformamide (DMF), N,N-dimethylacetamide (DMA), dimethylosulfoxide (DMSO), and hexamethylphosphoramide (HMPA).

A method where the concentration of Xn in mixture C is 0.01-0.05 mol/l.

A method where the mixture C is an organic solvent or a mixture of organic solvents immiscible with water as defined below, preferentially a mixture of n-heptane and ethyl acetate (in volume ratio v/v from 8:2 to 99:1):
esters with the general formula R₁CO₂R₂, where R₁/R₂=saturated and unsaturated aliphatic C₁-C₄ hydrocarbons and R₂=aromatic hydrocarbons or aromatic-aliphatic hydrocarbons (e.g. PhCH₂—), in particular ethyl acetate,
saturated, unsaturated, cyclic C₅-C₈ hydrocarbons, toluene (PhMe), xylenes (ortho-xylene, meta-xylene, para-xylene, and their mixtures),
liquid ethers with the general formula R₁OR₂ where R₁/R₂=saturated and unsaturated, acyclic and cyclic aliphatic C₁-C₄ hydrocarbons, in particular diethyl ether (Et₂O), diisopropyl ether (i-Pr₂O), tert-butyl methyl ether (t-BuOMe), and 2-methyl tetrahydrofuran (2-MeTHF).

A method where mixture D is aqueous solution of inorganic and/or organic acids containing chemical compounds or mixtures thereof from the list below:
citric acid, ethylenediaminetetraacetic acid (versenic acid, EDTA), trichloroacetic acid (CCl₃CO₂H),
hydrochloric acid (HCl), sulfuric acid (H₂SO₄), phosphoric acid (H₃PO₄), nitric acid (HNO₃), and sodium hydrogen sulfate (sodium bisulfate, NaHSO₄).

A method where the advantageous concentration of the mixture D is 0.05-0.2 mol/L.

A method where mixture E is an aqueous solution of inorganic salts containing transition metal cations of zinc, iron, cobalt, copper, and nickel, for example zinc chloride (ZnCl₂).

A method where the advantageous concentration of the mixture E is 0.05-0.1 mol/L.

A method where mixture F is an aqueous solution of weak inorganic and/or organic bases containing chemical compounds or mixtures thereof from the list below:
bicarbonates of metal cations, with the general formula MHCO₃, where M═Li, Na, K, Cs,
dihydrogen phosphates of metal cations, with the general formula M₂HPO₄, where M═Li, Na, K, Cs,
mono hydrogen phosphates of metal cations, with the general formula MH₂PO₄, where M═Li, Na, K, Cs,
salts of mono-or poly-carboxylic acids RCOOM, where M═Li, Na, K, Cs, and R=saturated and unsaturated aliphatic C₁-C₄ hydrocarbons and R₂=aromatic hydrocarbons or aliphatic-aromatic hydrocarbons,
nitrogen bases, with the general formula R1-N(R2)(R3), where R1/R3=H, saturated and unsaturated aliphatic C₁-C₄ hydrocarbons and R₂=aromatic hydrocarbons or aliphatic-aromatic hydrocarbons.

A method where the advantageous concentration of the mixture F is 0.05-0.1 mol/L.

A method where mixture G is an aqueous solution of inorganic salts containing cations of alkali metals (Li, Na, K, Cs) and alkaline earth metals (Be, Mg, Ca), advantageously sodium chloride, sodium sulfate or a mixture thereof.

A method where the advantageous concentration of the mixture G is 0.1-0.2 mol/L.

Advantages of the subject invention:
the final Xn product shows high purity (Xn content >95% by weight);
high process efficiency, allows Xn insulation with high efficiency;
the plant waste generated by the process according to the invention makes an excellent fertilizer for use in agriculture;
no need for toxic solvents such as halogenated alkanes;
recovery of solvents used in the technological process according to the invention, allows to reduce the amount of waste generated;
compact production scale, which also translates into less energy consumption.

Terms used above and in the patent description and claims have the following meanings:
Xanthohumol—interchangeably used Polish name ksantohumol.
Raw material of plant origin—these are outgrowths, plant material.
Outgrowths—a by-product of the hop extraction process.
Biologically active substance of plant origin—also called plant extract.
High purity—content of biologically active substance >95% by weight.
High efficiency—insulation efficiency from the material >80%.
Plant material—all parts of the hops plant and/or the product formed in the process of extracting hops with supercritical carbon dioxide.

### DESCRIPTION OF THE FIGURES

FIG. 1—demonstrates a block diagram of the process of isolation of xanthohumol from outgrowths.

FIG. 2—demonstrates the HPLC chromatogram of the plant extract.

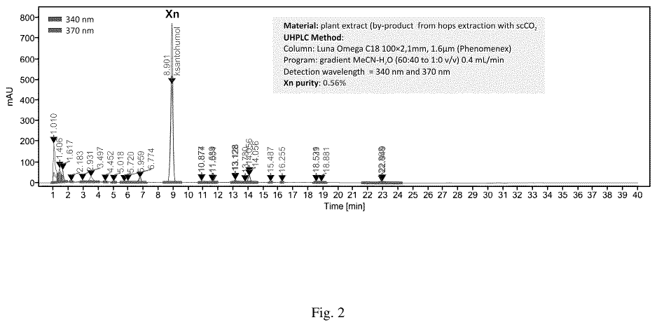

FIG. 3—demonstrates the HPLC chromatogram of xanthohumol after the final purification process.

The invention is illustrated by the following performance examples, which do not constitute a limitation of the invention.

### EXAMPLE 1

### Input Material

1) Green-gray dust and granules (outgrowths) with xanthohumol content of 0.1-50% by weight.
2) The plant material contains a large amount of non-polar and polar impurities that are difficult to remove by classical extraction processes (aqueous extraction with a non-water miscible organic solvent).

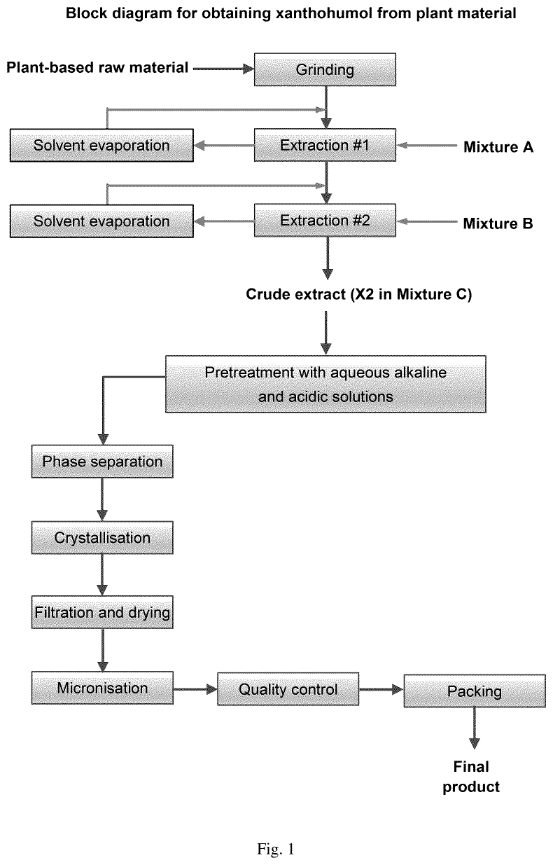

### Procedure for Obtaining Xn From Plant Material (FIG. 1):

### a) Pre-Isolation (Enrichment of Plant Raw Material)

The plant raw material is suspended in n-heptane and subjected to a continuous extraction process at 20-30° C. to remove non-polar impurities. Then, the extraction temperature is increased to 50-60° C. (some components of the plant raw material melt at this temperature), and the process is repeated. The heptane is evaporated, and the yellow residue is filtered through a filter using a press. The dry residue is transferred to an extraction reactor, ethyl acetate is added, and the process is repeated analogously to the n-heptane extraction, except that now the xanthohumol will accumulate in a flask on a rotary evaporator. At the end of the process, the content of xanthohumol in the yellowish precipitate is about 10-12% by weight.

The precipitate is dissolved in an appropriate amount of ethyl acetate-heptane mixture (9:1 v/v) to obtain a xanthohumol concentration of ~0.05 mol/L. The resulting dark solution is subjected to extraction in the presence of 0.2M citric acid solution, 0.05M ZnCl₂, 5% NaHCO₃, and brine. After drying the organic layer with anhydrous Na₂SO₄, and evaporating the organic solvent, a solid mixture with 20-25% xanthohumol content is obtained.

### b) Purification

The mixture is then dissolved in such an amount of ethyl acetate to obtain a concentration of xanthohumol of 0.05-0.1M; after that the solution is subjected to filtration and alkaline-acid flow extraction using a liquid-liquid separator. Specifically, the solution of xanthohumol in ethyl acetate is extracted against 0.05-0.1M NaOH solution, the organic layer is discarded, xanthohumol in the aqueous layer (in the form of sodium salt), is added to 0.05M-0.1M citric acid solution, which causes precipitation of xanthohumol, which is then extracted against ethyl acetate and dried with anhydrous Na₂SO₄. Ethyl acetate and brine are added to the residue. After draining the inorganic salt, most of the ethyl acetate is evaporated, and a minimal amount of n-heptane is added to initiate the precipitation of xanthohumol from the solvent mixture. The distillation process is continued until all the xanthohumol has precipitated, after which methanol is added to remove traces of ethyl acetate and the evaporation process continues. The yellow precipitate is drained off, washed with n-heptane, and dried under reduced pressure, obtaining xanthohumol as a light yellow powder with very high purity (299.5%, as determined by HPLC).

### Mixture A:

9. An organic solvent or mixture of organic solvents immiscible with water as defined below:
10. saturated, unsaturated, cyclic hydrocarbons C₅-C₈, toluene, xylenes (ortho-xylene, meta-xylene, para-xylene and their mixtures).

### Mixture B:

11. Polar organic solvent or mixture of polar organic solvents as defined below:
aliphatic alcohols with the general formula ROH, where R=C₁-C₄ aliphatic hydrocarbon, in particular MeOH, EtOH, and 2-propanol.
esters with the general formula R₁CO₂R₂, where R₁/R₂=saturated and unsaturated aliphatic C₁-C₄ hydrocarbons and R₂=aromatic hydrocarbons or aromatic-aliphatic hydrocarbons (e.g., PhCH₂). In particular, ethyl acetate.
liquid ethers with the general formula R₁OR₂ where R₁/R₂=saturated and unsaturated, acyclic and cyclic aliphatic C₁-C₄ hydrocarbons, in particular diethyl ether (Et₂O), diisopropyl ether (i-Pr₂O), tert-butyl methyl ether (t-BuOMe), and 2-methyl tetrahydrofuran (2-MeTHF),
liquid polar aprotic solvents N,N-dimethylformamide (DMF), N,N-dimethylacetamide, dimethylsulfoxide (DMSO), hexamethylphosphoramide (HMPA).

### Mixture C:

12. Organic solvent or mixture of organic solvents immiscible with water as defined below, preferentially a mixture of ethyl acetate/n-heptane 8:2-99:1 v/v)
esters with the general formula R₁O₂R₂, where R₁/R₂=saturated and unsaturated aliphatic C₁-C₄ hydrocarbons and R₂=aromatic hydrocarbons or aromatic-aliphatic hydrocarbons (e.g. PhCH₂). In particular, ethyl acetate.
saturated, unsaturated, cyclic C₅-C₈ hydrocarbons, toluene, xylenes (ortho-xylene, meta-xylene, para-xylene, and their mixtures).
liquid ethers with the general formula R₁OR₂ where R₁/R₂=saturated and unsaturated, acyclic and cyclic aliphatic hydrocarbons C₁-C₄, in particular diethyl ether (Et₂O), diisopropyl ether (i-Pr₂O), tert-butyl methyl ether (t-BuOMe), and 2-methyl tetrahydrofuran (2-MeTHF).

### Mixture D:

13. Aqueous solutions of inorganic and/or organic acids containing chemical compounds or mixtures thereof from the list below:
citric acid, ethylenediaminetetraacetic acid (versenic acid, EDTA), trichloroacetic acid (CCl₃CO₂H),
hydrochloric acid (HCl), sulfuric acid (H₂SO₄), phosphoric acid (H₃PO₄), nitric acid (HNO₃), and sodium hydrogen sulfate (sodium bisulfate, NaHSO₄).

### Mixture E:

14. An aqueous solution of inorganic salts containing transition metal cations of zinc, iron, cobalt, copper, and nickel; for example, zinc chloride (ZnCl₂).

### Mixture F:

15. Aqueous solutions of weak inorganic and/or organic bases containing chemical compounds or mixtures thereof from the list below:
bicarbonates of metal cations, with the general formula MHCO₃, where M═Li, Na, K, Cs
dihydrogen phosphates of metal cations, with the general formula M₂HPO₄, where M═Li, Na, K, Cs
Mono hydrogen phosphates of metal cations, with the general formula MH₂PO₄ where M═Li, Na, K, Cs
salts of mono-or poly-carboxylic acids RCOOM, where M═Li, Na, K, Cs, and R=saturated and unsaturated aliphatic hydrocarbons C₁-C₄ and R₂=aromatic hydrocarbons or aliphatic-aromatic hydrocarbons.
nitrogen bases, with the general formula R1-N(R2)(R3), where R1/R3=H, saturated and unsaturated aliphatic hydrocarbons C₁-C₄ and R₂=aromatic hydrocarbons or carbohydrates R.

### Mixture G:

16. An aqueous solution of inorganic salts containing cations of alkali metals (lithium, Li, Na, K, Cs) and alkaline earth metals (beryllium, Mg, Ca). Preferentially sodium chloride, sodium sulfate, or a mixture of these.

### Mixture I:

17. Aqueous solutions of strong inorganic and/or organic bases containing chemical compounds or mixtures thereof from the list below:
alkali metal hydroxides, with the general formula MOH, where M═Li, Na, K, Cs orthophosphates of metal cations, with the general formula M₃PO₄, where M═Li, Na, K, and Cs,
strong organic bases, e.g., 1,8-Diazabicyclo(5.4.0)undec-7-ene (DBU), NR₁R₂R₃ where R—R₁₃═H, C₁-C₆ aliphatic hydrocarbon.

### Stage I

18. A weighed amount of plant-derived raw material (raw material (a)—Xn content 0.1-2.0% by weight; raw material (b) Xn content 10-40% by weight) is placed in a steel extractor equipped with mechanical stirrers, a reflux condenser and a system of dedicated filters. A measured amount of mixture A is then added. The extractor contents are continuously extracted under an inert gas atmosphere at 20-90° C. (preferentially 25-50° C.) for 2-96 h (preferentially 12-36 h). Mixture A and soluble fractions of the plant's raw material are taken through a suitable system of dedicated flow-through filters for evaporation. The evaporated solvent is returned to the extractor, and the solid residue X1 is waste.

### Stage 2

19. Mixture A is removed from the extractor, and mixture B is added to the constant mass. The extractor contents are continuously extracted in an inert gas atmosphere at 20-90° C. (preferentially 20-50° C.) for 0.5-24 h (preferentially 6-12 h). Mixture B and soluble fractions of the plant material are taken through a suitable ceramic flow-through filter system for evaporation. The B mixture is evaporated and the solid residue X2 is the raw product for further processing. After the extraction is completed, the extractor contents are shunted to the filter press. The filtrate after evaporation of mixture B is attached to X2 (a) the solid residue Xn content is 5-20% by weight (when raw material (a) was used) or 20-80% (when raw material (b) was used). The solid residue from the filter press is waste X3, which can be used as fertilizer.

### Stage 3

20. The weighed amount of X2 is dissolved in mixture C to obtain a concentration of a) Xn 0.005-0.5 mol/l (preferentially 0.01-0.05 mol/l), b) the same as a). The resulting solution X2 in mixture C is subjected to a process of successive single or multiple extractions against mixture D of 0.005-1.0 mol/l (preferentially 0.05-0.2 mol/l), mixture E of 0.005-0.2 mol/l (preferentially 0.05-0.1 mol/1), mixture F with a concentration of 0.005-1.0 mol/l (preferentially 0.05-0.1 mol/l), and mixture G with a concentration of 0.05-1.0 mol/l (preferentially 0.1-0.2 mol/l) to obtain solution H (Xn content in the dry weight is 20-25% by weight when raw material (a) was used and 30-90% by weight when raw material (b) was used). The extraction process is performed at 10-50° C. (preferentially 10-30° C.), under an inert gas atmosphere.

### Stage 4

At this stage of isolation and purification of Xn, a reversible process of formation of very water-soluble Xn salts (phenolates) is used, which, after neutralization with an aqueous acid solution from the D-mixture, results in a suspension of Xn in water. Xn is practically insoluble in water (1.3 mg/L at 23° C., J. Agric. Food Chem. 1999, 47, 2421-2428).
21. The H solution is set to an Xn content of 0.01-0.1 mol/L (preferentially 0.05-0.10 mol/L), after which the H solution is subjected to an alkaline-acid extraction process in flow using a liquid-liquid separator.
22. Two streams of solutions are fed into the flow reactor-solution H (Xn in an organic solvent immiscible with water) and mixture I.
23. The flow reactor is connected directly to the liquid-liquid separator. The organic stream from the separator is waste X4, and the aqueous stream is subjected to a neutralization process with a mixture D. The extraction process is carried out at a temperature of 2-35° C. (preferentially 20-25° C.), in an inert gas atmosphere.
24. The result is a suspension of Xn in water, which is subjected to (a) filtration, (b) centrifugation or (c) extraction with a mixture of C.
25.After the C extraction process, the organic phase is washed with a mixture of G (preferentially sodium chloride or sodium sulfate) at a concentration of 0.05-1.0 mol/L (preferentially 0.1-0.2 mol/L) and then dried with molecular sieves or anhydrous sodium sulfate (Na₂SO₄), magnesium sulfate (MgSO₄) or their mixture of the listed drying agents. After draining the drying agent, we obtain J-solution (Xn content in the dry mass is 80-90% by weight).
26. After the filtration (a) or centrifugation (b), the Xn precipitate is dissolved in the C mixture and subjected to analogous operations as after the process (c).

### Stage 5

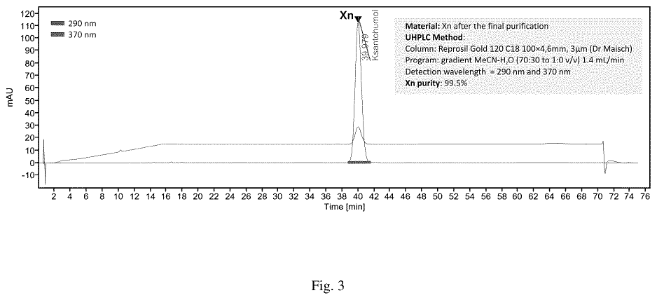

At this stage, a final purification is performed to obtain Xn of high pharmaceutical purity (Xn content in dry weight is ≥99.5%, as determined by HPLC).
27. A measured amount of Mixture A, in which Xn is sparingly soluble (preferentially C7-C8 saturated hydrocarbons, preferably n-heptane), is added to solution J, and the volatile fractions are evaporated until Xn precipitation is initiated. The precipitated Xn is drained off and washed with mixture A at 0-40° C. (preferably 15-25° C.). The filtrate constitutes the waste X5. The precipitate is macerated with mixture C (preferentially a mixture of aliphatic hydrocarbons and ethers).
28. The insoluble precipitate is re-dried, and the filtrate is waste X6. The residue is dried at 15-25° C. under reduced pressure. The dry residue is dissolved in mixture B (preferentially in aliphatic alcohol), and the volatile fractions are evaporated to dryness under reduced pressure at 15-35° C. (preferentially 25-30° C.).
29. After vacuum drying at 15-25° C., the final product is obtained as a light yellow powder (Xn content is ≥99.5%, as determined by HPLC, FIG. 3).

1. A method of isolation and purification of xanthohumol by extraction from plant material characterized in that:
an appropriate amount of plant material is weighed and then a measured amount of mixture A is added, where the contents of the extractor are subjected to a continuous extraction process under an inert gas atmosphere at 20-90° C. for 2-96 h, mixture A and the soluble fractions of the plant material are taken through a suitable ceramic flow filter system for evaporation, and the evaporated solvent is returned to the extractor, and the solid residue X1 is waste;
mixture A is removed from the extractor, and mixture B is added to the solid mass, where the extractor contents are subjected to a continuous extraction process under an inert gas atmosphere at 20-90° C. for 0.5-24 h; mixture B and the soluble fractions of the plant raw material are taken through a suitable ceramic flow filter system for evaporation, mixture B is evaporated and the solid residue X2 is the raw product for further processing; after the extraction is completed, the extractor contents are swept to a filter press and the filtrate after the evaporation of mixture B is attached to X2, the solid residue from the filter press is the waste X3;
the weighed amount of X2 is dissolved in mixture C to obtain a concentration of Xn 0.005-0.5 mol/l, the resulting solution of X2 in mixture C is subjected to a process of successive single or multiple extractions against mixture D with a concentration of 0.005-1.0 mol/l, mixture E with a concentration of 0.005-0.2 mol/l, mixture F with a concentration of 0.005-1.0 mol/l and mixture G with a concentration of 0.05-1.0 mol/l to obtain solution H, the extraction process is carried out at a temperature of 10-50° C., preferably 10-30° C., in an inert gas atmosphere;
in this step of isolation and purification of Xn, a reversible process of formation of very water-soluble Xn salts (phenolates) is used, which, after neutralization with an aqueous acid solution from the D-mixture, results in a suspension of Xn in water, the H-solution is set to an Xn content of 0.01-0.1 mol/l (preferentially 0.05-0.10 mol/l), after which the H solution is subjected to an alkaline-acid extraction process in the flow using a liquid-liquid separator, two streams of solutions are fed into the flow reactor-the H solution and the I mixture, the flow reactor is connected directly to the liquid-liquid separator, the organic stream coming out of the separator is waste X4, and the water stream is subjected to neutralization with mixture D, the extraction process is carried out at a temperature of 2-35° C., preferentially 20-25° C., in an inert gas atmosphere;
a suspension of Xn in water is obtained, which is subjected to (a) filtration, (b) centrifugation, or (c) extraction with mixture C; the organic phase after the extraction process is washed with mixture G, preferably sodium chloride or sodium sulfate, of 0.05-1.0 mol/l, preferably 0.1-0.2 mol/l, and then dried with molecular sieves or anhydrous sodium sulfate, magnesium sulfate or their mixture of the said drying agents; after the drying agent is drained off, solution J is obtained (Xn content in dry weight is 80-90% by weight); after filtration (a) or centrifugation (b), the Xn precipitate is dissolved in mixture C and subjected to analogous processes as after process (c);
a measured amount of Mixture A is added to solution J in which Xn is sparingly soluble (preferentially C7-C8 saturated hydrocarbons, preferably n-heptane), and the volatile fractions are evaporated until Xn precipitation is initiated, the precipitated Xn is drained and washed with Mixture A at 0-40° C. (preferably 15-25° C.), the filtrate constitutes waste X5, the precipitate is macerated with Mixture C, insoluble precipitate is re-drained and the filtrate is waste X6, the precipitate is dried at 15-25° C. under reduced pressure, the dry precipitate is dissolved in mixture B and the volatile fractions are evaporated to dryness under reduced pressure at 15-35° C., preferably 25-30° C., after vacuum drying at 15-25° C., the final product is obtained in the form of a light yellow powder (Xn content ≥99.5% according to HPLC).

2. A method according to claim 1, characterized in that the extraction in an inert gas atmosphere is advantageously at 25-50° C. for 6-36 h.

3. A method according to claim 1, characterized in that the mixture A is saturated, unsaturated, or cyclic C₅-C₈ hydrocarbons, toluene, xylenes (ortho-xylene, meta-xylene, para-xylene and mixtures thereof).

4. A method according to claim 1, characterized in that the residue X1, X2, or X3 is xanthohumol.

5. A method according to claim 1, characterized in that the mixture B is a polar organic solvent or a mixture of polar organic solvent i.e.:
aliphatic alcohols with the general formula ROH, where R=C₁-C₄ aliphatic group, in particular methyl alcohol (MeOH), ethyl alcohol (EtOH), 2-propanol (i-PrOH),
esters with the general formula R₁CO₂R₂, where R₁/R₂=saturated and unsaturated aliphatic C₁-C₄ hydrocarbons and R₂=aromatic hydrocarbons or aromatic-aliphatic hydrocarbons (e.g. PhCH₂—), in particular ethyl acetate,
liquid ethers with the general formula R₁OR₂ where R₁/R₂=saturated and unsaturated, acyclic and cyclic aliphatic hydrocarbons C₁-C₄, in particular in particular diethyl ether (Et₂O), diisopropyl ether (i-Pr₂O), tert-butylmethylether (t-BuOMe), and 2-methyltetrahydrofuran (2-MeTHF),
liquid polar aprotic solvents such as N,N-dimethylformamide (DMF), N,N-dimethylacetamide (DMA), dimethylosulfoxide (DMSO), and hexamethylphosphoramide (HMPA).

6. A method according to claim 1 characterized in that the concentration of Xₙ in mixture C is 0.01-0.05 mol/l.

7. A method according to claim 1 characterized in that the mixture C is an organic solvent or a mixture of organic solvents immiscible with water as defined below, preferentially a mixture of n-heptane and ethyl acetate (in volume ratio v/v from 8:2 to 99:1):
esters with the general formula R₁CO₂R₂, where R₁/R₂=saturated and unsaturated aliphatic C₁-C₄ hydrocarbons and R₂=aromatic hydrocarbons or aromatic-aliphatic hydrocarbons (e.g. PhCH₂—), in particular ethyl acetate,
saturated, unsaturated, cyclic C₅-C₈ hydrocarbons, toluene (PhMe), xylenes (ortho-xylene, meta-xylene, para-xylene and their mixtures),
liquid ethers with the general formula R₁OR₂ where R₁/R₂=saturated and unsaturated, acyclic and cyclic aliphatic C₁-C₄ hydrocarbons, in particular diethyl ether (Et₂O), diisopropyl ether (i-Pr₂O), tert-butylmethylether (t-BuOMe), and 2-methyltetrahydrofuran (2-MeTHF).

8. A method according to claim 1, characterized in that the mixture D is aqueous solutions of inorganic and/or organic acids containing chemical compounds or mixtures thereof from the list below: citric acid, ethylenediaminetetraacetic acid (versenic acid, EDTA), trichloroacetic acid (CCl₃CO2H), hydrochloric acid (HCl), sulfuric acid (H₂SO₄), phosphoric acid (H₃PO₄), nitric acid (HNO₃), sodium hydrogen sulfate (sodium bisulfate, NaHSO₄.

9. A method according to claim 1, characterized in that the advantageously the concentration of the mixture D is 0.05-0.2 mol/L.

10. A method according to claim 1, characterized in that the mixture E is an aqueous solution of inorganic salts containing transition metal cations of zinc, iron, cobalt, copper and nickel, for example zinc chloride (ZnCl₂).

11. A method according to claim 1, characterized in that the advantageously the concentration of the mixture E is 0.05-0.1 mol/L.

12. A method according to claim 1, characterized in that the mixture F is an aqueous solution of weak inorganic and/or organic bases containing chemical compounds or mixtures thereof from the list below:
bicarbonates of metal cations, with the general formula MHCO₃, where M═Li, Na, K, Cs,
dihydrogen phosphates of metal cations, with the general formula M₂HPO₄, where M═Li, Na, K, Cs,
monohydrogen phosphates of metal cations, with the general formula MH₂PO₄, where M═Li, Na, K, Cs,
salts of mono- or poly-carboxylic acids RCOOM, where M═Li, Na, K, Cs, and R=saturated and unsaturated aliphatic C₁-C₄ hydrocarbons and R₂=aromatic hydrocarbons or aliphatic-aromatic hydrocarbons,
nitrogen bases, with the general formula R1-N(R2)(R3), where R1/R3=H, saturated and unsaturated aliphatic C₁-C₄ hydrocarbons and R₂=aromatic hydrocarbons or aliphatic-aromatic hydrocarbons.

13. A method according to claim 1, characterized in that the advantageously the concentration of the mixture F is 0.05-0.1 mol/L.

14. A method according to claim 1, characterized in that the mixture G is aqueous solutions of inorganic salts containing cations of alkali metals (Li, Na, K, Cs) and alkaline earth metals (Be, Mg, Ca), advantageously sodium chloride, sodium sulfate or a mixture thereof.

15. A method according to claim 1, characterized in that the advantageously the concentration of the mixture G is 0.1-0.2 mol/L.
